(19) United States
(12) Patent Application Publication
(10) Pub. No.: US 2022/0160524 A1
(43) Pub. Date: May 26, 2022

(54) HIGH DENSITY DISTANCE SENSOR ARRAY ALTERNATIVE TO SURFACE ELECTROMYOGRAPHY FOR THE CONTROL OF POWERED UPPER LIMB PROSTHESES

(71) Applicant: GAIA TECH, L.L.C., Murray, UT (US)

(72) Inventors: Nicholas Steven WITHAM, Murray, UT (US); Devan James ANDERSON, Murray, UT (US); Farshad MOGHARRABI, Salt Lake City, UT (US); Rami SHORTI, Salt Lake City, UT (US)

(21) Appl. No.: 17/602,382

(22) PCT Filed: Apr. 8, 2020
(86) PCT No.: PCT/US2020/027314

§ 371 Date: Oct. 8, 2021

Related U.S. Application Data
(60) Provisional application No. 62/830,837, filed on Apr. 8, 2019.

(51) Int. Cl. A61F 2/72; A61B 5/00; A61F 2/58; A61F 2/80; G06F 3/01; G06F 3/0346; B25J 15/00

(52) U.S. Cl. CPC A61F 2/72; A61B 5/4851; A61F 2/583; A61F 2/80; A61F 2002/704; G06F 3/017; G06F 3/011; G06F 3/0346; B25J 15/0009; A61F 2/586

(57) ABSTRACT

Systems and methods for a wearable sensor system including a compressible material, a two-dimensional array of distance sensors, a support structure, and a controller. The compressible material is positionable relative to a tissue surface and the two-dimensional array of distance sensors is configured relative to the compressible material to detect compressive deformations of the compressible material. The support structure is configured to hold the compressible material in place relative to the tissue surface such that muscle movements at the tissue surface cause the compressive deformations of the compressible material and is also configured to restrict movement of the two-dimensional array during the muscle movements. The controller is configured to receive a signal from the two-dimensional array indicative of the compressive deformation of the compressive material at a location of each distance sensor

### RELATED APPLICATIONS

This application claims the benefit of U.S. Provisional Patent Application No. 62/830,837, filed Apr. 8, 2019, and entitled “HIGH DENSITY DISTANCE SENSOR ARRAY ALTERNATIVE TO SURFACE ELECTROMYOGRAPHY FOR CONTROL OF POWERED UPPER LIMB PROSTHESIS,” the entire contents of which are incorporated herein by reference.

### BACKGROUND

The present disclosure relates to methods and system for detecting and monitoring movements of a hand. More particularly, in some implementations, the present disclosure relates methods and systems for detecting muscle movements for controlling a powered prosthesis.

### SUMMARY

Despite the technological achievements of modern-day prostheses, the average person with upper limb amputation (ULA) is unable to gain a significant level of prosthetic embodiment. In general, a level of embodiment can be described as how well a person projects and attaches their sense of self to their body, other individuals, objects, and concepts. Prosthetic embodiment in particular has to do with the extent that a person identifies a prosthetic device as part of their self-identity and body. Usually, in literature, the amount of time an amputee wears a prosthesis is used as a correlate for a level of their prosthetic embodiment. However, for most people with ULA, there is very little prosthetic embodiment.

In some implementations, the present disclosure provides methods and systems for a wearable two-dimensional, high-density array of distance sensors for use in controlling a prosthesis or an animatronic device based on sensed muscle movements along a tissue surface. For example, in some implementations, the operation of a powered prosthetic hand is controlled based on changes in the shape of the forearm due to movements of the forearm muscles. As a user contracts the muscles in his/her forearm, there is a change in distance between the muscles and one or more of the sensors of the array of distance sensors positioned on the forearm surface. The distance sensors detect the change in distance and detect where on the arm the change occurred. A control signal based on the detected sensor information is transmitted to the prosthetic or animatronic hand, which then accomplishes the desired motion. This control device and methodology will enable a patient to perform real-time, direct, robust, and simultaneous control of multiple degrees of freedom.

In other implementations, the two-dimensional array of distance sensors is configured for placement on a different muscle surface. For example, in some implementations, the two-dimensional array of distance sensors is configured for placement on a leg surface and outputs signals indicative of changes in the surface of the leg due to movements of the leg muscles. Those output signals are then used, in some implementations, to operate the actuators of a powered leg and/or foot prosthesis. Similarly, in other implementations, the two-dimensional array of distance sensors is configured for placement on a chest surface and outputs signals indicative of changes in the surface of the chest due to movements of the pectoral muscles. Those output signals are then used, in some implementations, to operate the actuators of a powered arm prosthesis.

In still other implementations, the two-dimensional array of distance sensors is configured to monitor muscle movements in order to control other non-prosthetic system. For example, in some implementations, forearm muscle movements are monitored by the two-dimensional array of distances sensors in order to determine movements and/or placement of a user's hand for controlling a virtual reality (VR) or augmented reality (AR) systems.

In some embodiments, the invention provides a wearable sensor system including a compressible material, a two-dimensional array of distance sensors, a support structure, and a controller. The compressible material is positionable relative to a tissue surface and the two-dimensional array of distance sensors is configured relative to the compressible material to detect compressive deformations of the compressible material. The support structure is configured to hold the compressible material in place relative to the tissue surface such that muscle movements at the tissue surface cause the compressive deformations of the compressible material and is also configured to restrict movement of the two-dimensional array during the muscle movements.

The controller is configured to receive a signal from the two-dimensional array indicative of the compressive deformation of the compressive material at a location of each distance sensor and to determine a gesture operation based on the signal.

Other aspects of the invention will become apparent by consideration of the detailed description and accompanying drawings.

### BRIEF DESCRIPTION OF THE DRAWINGS

FIG. 1A is a block diagram of a prosthesis control system including a two-dimensional array of distance sensors and a multiplexer configured to generate a serialized output signal indicative of distances sensed by each sensor in the two-dimensional array, in accordance with some embodiments.

FIG. 1B is another block diagram of an example of the prosthesis control system of FIG. 1A.

FIG. 1C is a block diagram of a prosthesis controller of a prosthesis control system, in accordance with some embodiments.

FIG. 1D is a circuit diagram of the prosthesis control system of FIG. 1A including a filtering circuit for signal conditioning.

FIG. 2A is a block diagram of a prosthesis control system including a two-dimensional array of distance sensors and a de-multiplexer configured to selectively control which sensors are energized in order to generate a serialized output signal indicative of distances sensed by each sensor in the two-dimensional array, in accordance with some embodiments.

FIG. 2B is a circuit diagram of the prosthesis control system of FIG. 2A including a filtering circuit for signal conditioning.

FIG. 2C is a graph of the output signal of the two-dimensional array of distance sensors in the prosthesis control system of FIG. 2A before and after applying the filtering of the filtering circuit of FIG. 2B.

FIG. 3 is a perspective view of three examples of commercially available prosthetic hands that may be used with the prosthesis control system of FIG. 1A, 1B, or 2A, in accordance with some embodiments.

FIG. 4A is a cross-sectional view of distance sensors in the two-dimensional array positioned relative to a compressible layer, in accordance with some embodiments.

FIG. 4B is a perspective view of an example of the compressible layer equipped with a plurality of distance sensors positioned to form a two-dimensional array, in accordance with some embodiments.

FIG. 5 is a cross-sectional view of the various layers of a wearable control system, such as the prosthesis control system of FIG. 1A, 1B, or 2A, relative to the tissue surfaces of a user, in accordance with some embodiments.

FIG. 6A is an overhead view of an example of the flexible incompressible support layer of the wearable control system of FIG. 5.

FIG. 6B is an overhead view of an example of a flexible printed circuit board (PCB) layer of the wearable control system of FIG. 5 equipped with a two-dimensional array of light-intensity distance sensors.

FIG. 6C is a perspective view of an example of a wearable control system of FIG. 5 including the flexible incompressible support layer of FIG. 6A and the flexible PCB layer of FIG. 6B positioned relative to a prosthetic hand and a user's arm before the wearable control system is secured to the user's arm.

FIG. 6D is a perspective view of the wearable control system of FIG. 6C secured to the user's arm.

FIG. 7 is a flowchart of a method for controlling an actuator using a control system, such as illustrated in FIG. 2A, with a de-multiplexer configured to generate a serialized output signal by selectively controlling which sensors are energized.

FIG. 8 is a flowchart of a method for controlling an actuator using a control system, such as illustrated in FIG. 1A, with a multiplexer configured to generate a serialized output signal from the output of multiple simultaneously energized sensors.

FIG. 9 is a flowchart of a method for calibrating a control system such as the prosthetic control system of FIG. 1A, 1B, or 2A, in accordance with some embodiments.

FIG. 10 is a block diagram of a system configured to control an actuator of a powered hand prosthesis based on movements of forearm muscles measured by a two-dimensional array of distance sensors, in accordance with some embodiments.

FIG. 11 is a block diagram of a system configured to control an actuator of a powered arm prosthesis based on movements of pectoral muscles measured by a two-dimensional array of distance sensors, in accordance with some embodiments.

FIG. 12 is a block diagram of a system configured to control an actuator of a powered leg or foot prosthesis based on movements of leg muscles measured by a two-dimensional array of distance sensors, in accordance with some embodiments.

FIG. 13 is a block diagram of a system configured to control an actuator of a virtual reality (VR) or augmented reality (AR) system using extremity movements determined based on muscle surface movements measured by a two-dimensional array of distance sensors, in accordance with some embodiments.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

As noted above, the average person with ULA is unable to gain a significant level of prosthetic embodiment. However, the present disclosure enables great strides with osseointegration and neural prostheses, which can restore sensation by utilizing slanted electrode arrays. For the 96% of amputees who are not supported by Veterans Affairs, their upper-limb prosthesis may cost around $35,000-$75,000 with very little insurance coverage. The most effective prostheses in this price range use surface electromyography (sEMG) sensors and have less than a third of the degrees of freedom (DoF), or unique motions, of their natural counterpart. This is because every unique motion adds cost, bulk, and complexity to a prosthesis system. Beyond the problem of low resolution, sEMG requires filtering and excessive calibration and cannot differentiate between changes in muscle length, size, or speed of contraction. Also, these sEMG systems require excessive maintenance. Amputees must take time off from work to go to occupational therapists, physical therapists, and prosthetists or orthotists to keep their artificial limb working properly for the rest of their life. Due to these negative issues with upper-limb prostheses, 44-73% of people with ULA (based on level of amputation) do not use any prosthesis and they often feel disillusioned with the unintuitive expensive prostheses available to them.

The present disclosure provides cost efficient components for an electronically controllable prostheses at about 1/10th of the cost and circuit complexity of sEMG systems. Also, geometric anatomical measurements are made for the control of prosthesis hand geometry. Unlike EMG technology measurements, the geometric measurements enabled by the present disclosure can be directly related to muscle force, length, and velocity as measured using the high-density array of distance sensors. This provides for control of several degrees of freedom simultaneously where prosthesis motor actuation can vary in speed and position for unique motions of the prosthetic hand. For example, a person using a prostheses that is controlled based on the present disclosure could control the prosthesis intuitively and could play the piano, which is not presently available to a person with a ULA. The present disclosure provides simultaneous control of position, velocity, and force of prosthesis movement and gestures as intended and controlled by the user of the prosthesis. These improvements in controlling a prosthesis further increase a user's prosthetic embodiment and reduction in phantom pain. Furthermore, the present disclosure provides intuitive and easy to follow calibration of the prosthetic controller.

People with below-elbow amputations would benefit from the ability to consistently use their existing anatomy to restore hand dexterity with higher functionality as provided by the present disclosure. They would experience higher functionality than provided by earlier prosthetic control systems. For example, the present disclosure enables real-time, direct, and robust control. The natural, intuitive, and comfortable interface provides simultaneous control of multiple DoFs. In some embodiments a simple calibration method and system requires relatively limited training. The present distance sensor array prosthesis control device is designed for daily use, for use over long periods of time, and for a variety of indoor and outdoor environments. It is non-invasive and can be worn without adhesives. Users may include below elbow upper limb amputees that benefit from control of a below elbow prosthesis.

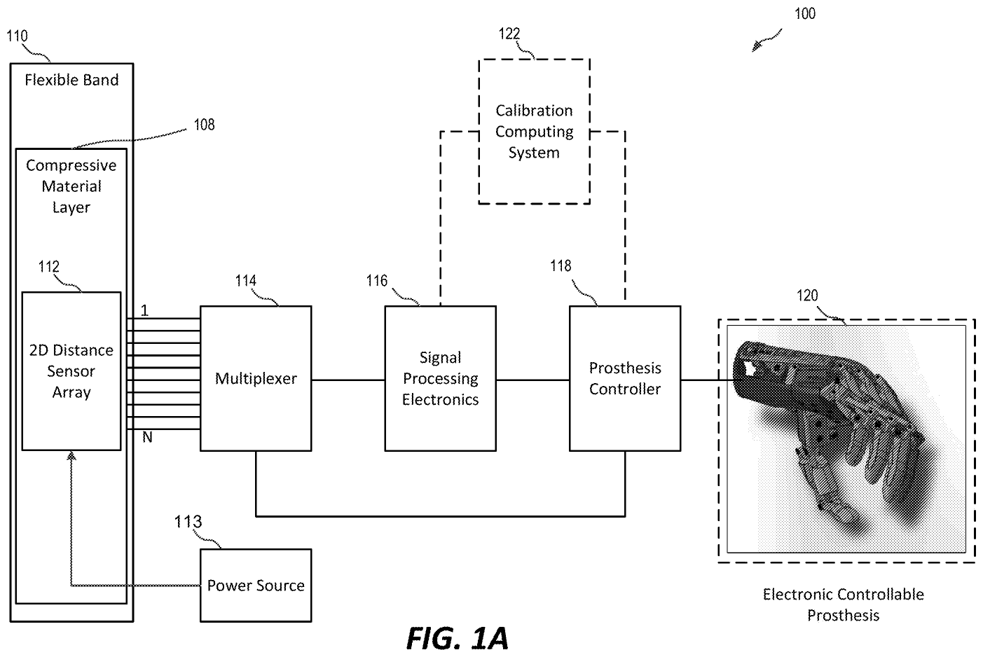

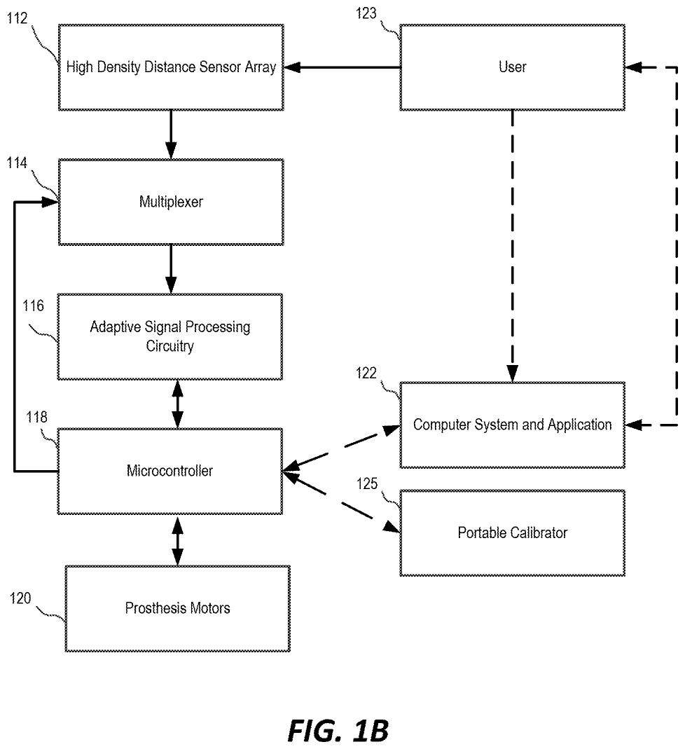

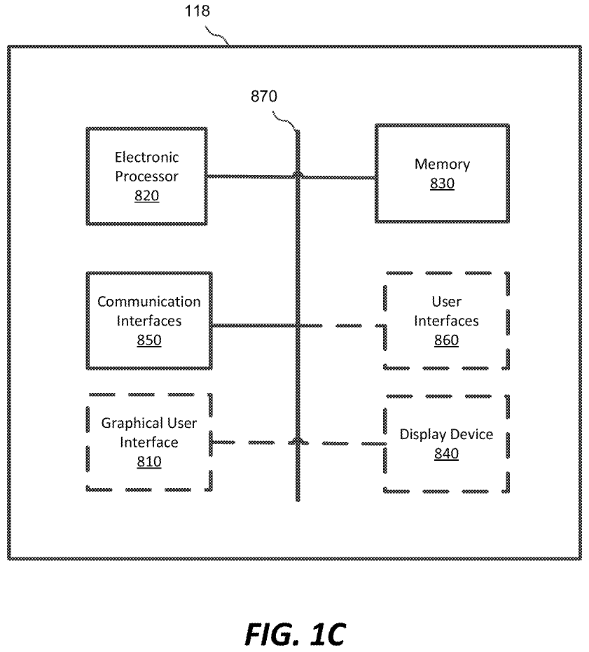

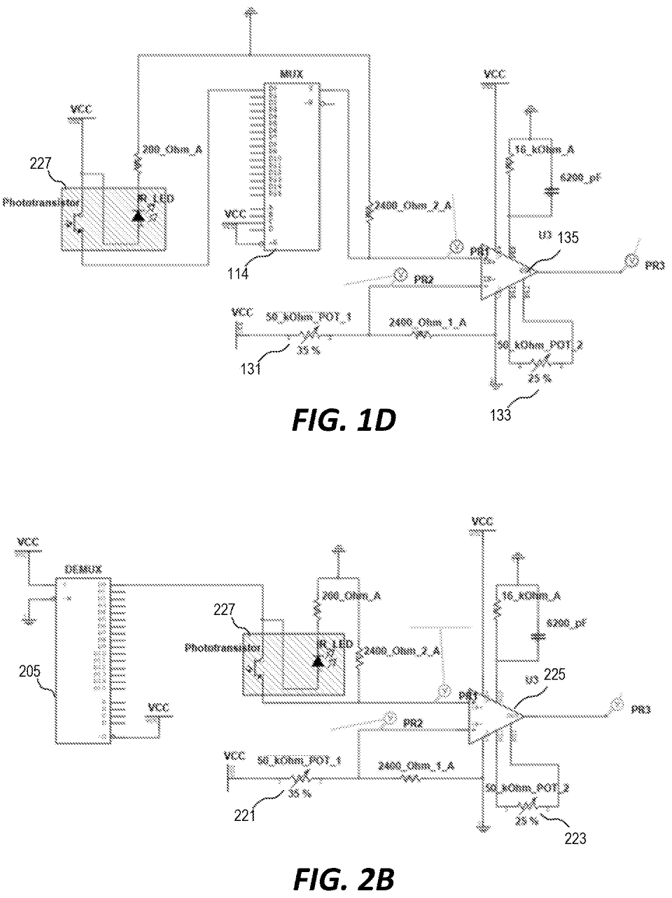

FIG. 1A is a block diagram of a prosthesis control system including an array of distance sensors. FIG. 1A includes a prosthesis control system 100 comprising a compressive material 108, a flexible band 110, distance sensor array 112, a multiplexer 114, a signal processing circuitry 116, a prosthesis controller 118, a prosthesis device 120, and a calibration computing system 122. The prosthesis controller 118 controls the prosthesis device 120 based on output from the distance sensor array 112. In some embodiments, the compressive material 108, the flexible band 110, the distance sensor array 112, the multiplexer 114, the signal processing circuitry 116, and the prosthesis controller 118 are components of an integrated wearable prosthesis control assembly for interaction between a person wearing the integrated assembly and a prosthesis including a hand that is also worn by the user such as described in further detail in the examples below.

In some implementations, the distance sensor array 112 includes a two-dimensional array of light intensity sensors disposed on, attached to, or otherwise held in place relative to a user's body part (e.g., the user's forearm) by the flexible band 110. The flexible band 110 may be a wearable band and may be wrapped or positioned around a user's forearm over the muscles that will be activated by the user and sensed by the distance sensor array 112 when the user controls the prosthesis device 120. In other implementations, the flexible band 110 is provided as a part of a wearable control and socket system that is further configured to selectively attach the prosthesis device 120 to a user's limb. In some embodiments, the flexible band 110 may be fastened, for example, by a double D ring loop strap, Velcro, or another suitable fastener. In some embodiments, the flexible band 110 may be made of a polymer material and may be injection molded, 2D printed, 3D printed, laser-cut, or die-pressed, for example. In some embodiments, the distance sensor array 112, the multiplexer 114, the signal processing unit 116, and prosthesis controller 118 may be integrated into one wearable band prosthesis control assembly.

In the example of FIG. 1A, the sensors of the distance sensor array 112 and the flexible band 110 are selectively positioned over the muscles of the forearm such that a 2-dimensional or 3-dimensional image or mapping of positions of the muscles may be obtained from the distance sensor measurements. The compressive material 108 may be disposed between the distance sensor array 112 and the user's forearm. The compressive material 108 helps to prevent motion of the distance sensor array 112 due to underlying muscle movement or changes in muscle thickness so that relative distance to the distance sensors 112 caused by the muscle thickness changes can be detected.

In some embodiments, the 2D array of distance sensors includes a plurality of light intensity sensors or LIDAR sensors. In some such implementations, each sensor includes a light source (e.g., a light emitting diode (LED)) and a light sensor (e.g., a phototransistor or photodiode). Light is emitted from the light source into the compressive material layer and reflected back towards the sensors where it is sensed by the phototransistor. The output signal of the phototransistor is indicative of a distance (or a change in distance) between the sensor and the tissue surface. In some implementations (as described in further detail below), the compressive material layer includes a reflective surface or a reflective layer is positioned on the opposite side of the compressive material layer from the sensor array to reflect light emitted by the sensors back towards the sensors of the distance sensor array 112. In some implementations, the reflective material may be used to prevent skin color or moisture on the skin surface from affecting light intensity measurements by the distance sensor array 112. When the flexible band and compressive material are fixed to a user's forearm and a muscle or multiple muscles of the forearm are used to control intended hand movements, the distance sensor array 112 detects a change in distance between the muscles and the sensors of the array.

As noted above, the distance sensor array 112 may comprise a high-density array of light intensity sensors. The density of the sensors used in the array 112 may affect the resolution of muscle movement detection and the level of control or fineness of articulation in the movements of prosthesis parts in the prosthesis device 120. The number of sensors in the array and the placement density of the sensors can vary in different implementations. In some examples, the sensor array may include 5 sensors, 250 sensors, or 1000 or more sensors. For example, the flexible band 110 may comprise a distance sensor array 112 comprising twenty-five sensors. The arrangement of the sensors of the array may vary depending the density of the array, the muscle group(s) and/or tissue surfaces on which the array with be positioned, and/or the positions of muscle motions that are used to map to movements or gestures of the prosthetic device 120. For example, in some embodiments, the sensors may be arranged in a triangular, rectangular, or radial two-dimensional (2D) grid. However, the disclosure is not limited to any specific number or arrangement of sensor elements in the distance sensor array 112.

In some embodiments, the distance sensor array 112 outputs analog signals. In the example of FIG. 1A, electrical power from a power source 113 is applied to all of the sensors in the array 112. The multiplexer 114 receives an output signal from each sensor in the sensor array 112 and indexes the sensors of the array one at a time based on a control input from the prosthesis controller 118. Each sensor's signal is serially output from the multiplexer 114 and may be conditioned by the signal processing electronics 116 to provide a well-resolved digital input to the prosthesis controller 118 and/or to the calibration computing system 122. For example, in some embodiments, the signal processing electronics include a voltage follower, a voltage subtracter, and a voltage amplifier to rectify the distance sensor signals and/or to limit the distance sensor signals to between about 0V and 5V. In some embodiments, the voltage subtracter and voltage amplifier may each have digital potentiometers or rheostat that allows the prosthesis control system to automatically rectify the signal (e.g., between 0V and 5V). In other examples, the circuit may be customized to the tolerances of the design or manually adjusted analogue potentiometers could be utilized. In some embodiments, the prosthesis controller 118 and/or the calibration computing system may include a microcontroller/computer/control system that is coupled to the signal processing electronics via a USB C, HDMI, or other suitable wired communication mechanism. However, the prosthesis controller 118 is not limited with regard to any specific types of communication interfaces.

One example of the prosthesis controller 118 is shown in further detail in FIG. 1C and includes, among other things, an electronic processor 820, a computer-readable non-transitory memory 830, and a communication interface 850 that may be communicatively coupled via a bus 870. The communication interface 850 may be communicatively coupled to the multiplexer 114, the signal processing electronics 116, and the calibration computing system 122. The memory 830 stores instructions that are executed by the electronic processor 820 to control the multiplexer 114, read distance sensor signals from the distance sensor array 112, control the actuators/motors of the prosthesis device 120, and/or calibrate the prosthesis control system 100 according to the disclosure described herein. For example, the electronic processor 820 may move the fingers and/or thumb of the prosthesis device 120 based on information of the distance sensor signals. In some embodiments, the prosthesis controller 118 may be operable to control wrist movement of the prosthetic device 120. In some embodiments, the prosthesis controller 118 may include a user interface 860, a display device 840 and/or a graphical user interface 810 to receive input from a user and/or provide information to a user.

The prosthesis controller 118 may be communicatively coupled to the calibration computing system 122 for calibration of the prosthesis control system 100. The calibration computing system 122 may include among other things, an electronic processor, a memory, and a communication interface communicatively coupled to the prosthesis controller 118. The calibration computing system 122 may also include a user interface, a display device, and a graphical user interface for interaction with a user during calibration of prosthesis control system 100 (see description below). In some embodiments, the calibration computing device 122 may be a portable device, such as a laptop, a smart phone, or a dedicated device. In some embodiments the calibration computing device 122 and the prosthesis controller 118 may be integrated as one wearable device, for example, as integrated in or attached to the flexible band 110.

The prosthesis controller 118 determines which distance sensor locations and distance sensor signal values that are received from the array 112 correspond to which fingers and finger movements or gestures of the prosthesis device 120 through a calibration sequence. As described in further detail below, the calibration sequence facilitated by the calibration computing device 122 may instruct the user to flex the muscles that they believe correspond to specific hand motions that will be made by their prosthesis or as if the movements are made by their missing hand. In some embodiments, the calibration sequence consists of 32 (2⁵) gestures and may start with all fingers open then all fingers closed. From there the sequence proceeds through the binary (full close and full open) combinations of each finger (including thumb). However, the disclosure is not limited to any specific calibration gestures or sequence of calibration gestures and the calibration method may be based on the level of control implemented for the prosthesis device 120. In some embodiments, gestures may be repeated. For example, a calibration sequence may run through every gesture five times and average the distance sensor signals. Another step may include having the user use their muscles to intend to open and close their missing hand along with control of the prosthesis device 120 to get a reading of the relation of a sensed muscle diameter change to prosthesis finger position. This is because the relationship of diameter change to muscle length is not linear and not necessarily predictable.

FIG. 1B is another block diagram of the prosthesis control system including the array of distance sensors 112. Like the example of FIG. 1A, the system of FIG. 1B includes a high-density distance sensor array 112 configured to provide a signal output for each sensor to the multiplexer 114. The multiplexer 114 is controlled by the microcontroller 118 to produce a serialized signal output which is then provided from the multiplexer 114 to additional adaptive signal processing circuitry 116. The microcontroller 118 then operates the actuators and motors of the prosthesis 120 based on the processed distance signals. However, in addition to a computer system and application 122 for calibration, the example of FIG. 1B also includes a portable calibrator 125 and is further configured to show the relation of a user 123 wearing the wearable band prosthesis control assembly to other components of the system.

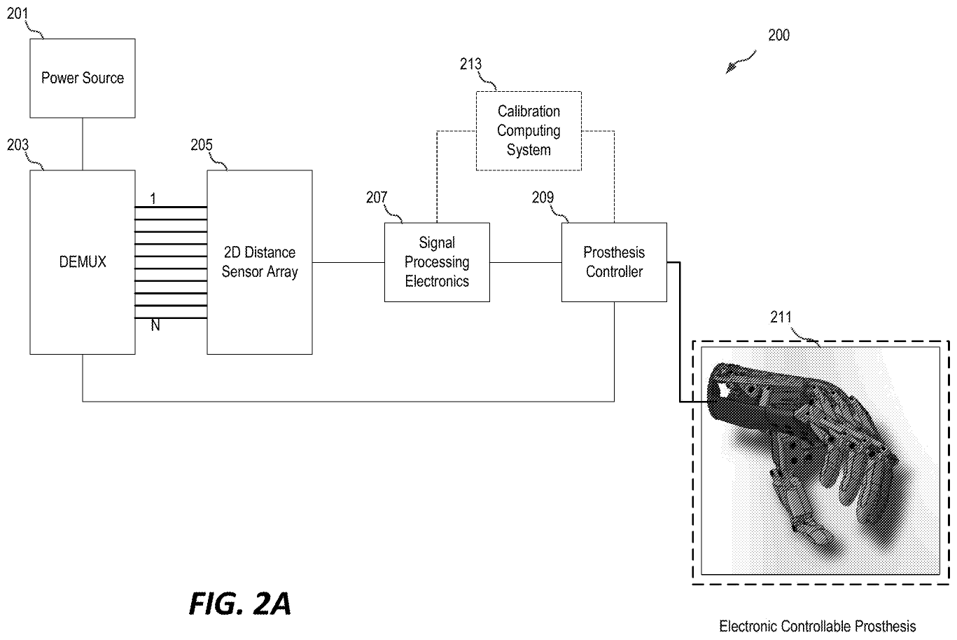

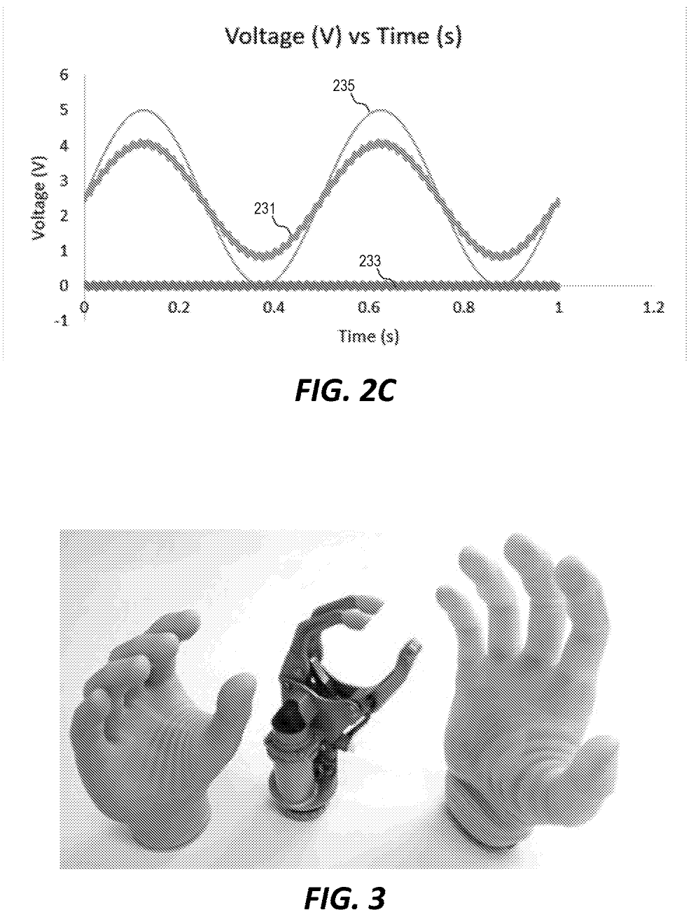

In the example of FIG. 1A, electrical power from the power source 113 is applied to all sensors in the sensor array 112 during operation of the system. However, due to the operation of the multiplexer 114, the signal from only a single sensor of the array 112 is included in the output signal of the multiplexer 114 at a given time. FIG. 2A illustrates an example of a control system 200 in which electrical power is further conserved by using a de-multiplexer (DEMUX) 203 to apply electrical power to only one sensor (or a subset of sensors) at a given time. A multiplexer (such as multiplexer 114 in the example of FIG. 1A) receives multiple input signals and provides one of those input signals as the output of the multiplexer based on the status of the control signal provided to the multiplexer. Conversely, a “demultiplexer” (such as DEMUX 203 in the example of FIG. 2A) receives a single input signal and applies that input signal to one or a plurality of output signal lines based on the status of the control signal input provided to the demultiplexer.

In the example of FIG. 2A, a power source 201 is coupled to the DEMUX 203 as the “input signal” for the DEMUX 203. Each output signal line of the DEMUX 203 is coupled to a different sensor in the 2D distance sensor array 205. Accordingly, depending on the control signal input provided to the DEMUX 203 (for example, by the prosthesis controller 209), the DEMUX 203 operates to connect the electrical power from the power source 201 to only one sensor of the sensor array 205 and, thereby, only one distance sensor of the array 205 is energized & operational at a given time. The outputs of all of the distance sensors in the array 205 are coupled to the same shared output line of the array 205 and, because only one distance sensor receives electrical power at a time, the shared output line produces a signal indicative of the distance sensed by the presently energized sensor.

In other words, in the example of FIG. 1A, a shared power source is coupled to all of the sensors in the array and the serialized output is produced by using the multiplexer 114 to selectively control which sensor output signal is connected to the output signal. In contrast, in the example of FIG. 2A, the serialized output signal is generated by using the DEMUX 203 to selectively control which individual sensor of the array 205 receives operating power.

Despite this different configuration, the output signal from the 2D sensor array 205 in the example of FIG. 2A is functionally equivalent to the output signal from the multiplexer 114 in the example of FIG. 1A. Accordingly, the system 200 of FIG. 2A similarly operates by providing the serialized output signal to signal processing electronics 207 and ultimately to the prosthesis controller 209, which, in turn, operates the motors and actuators of the prosthesis device 211 based on the serialized distance signal. The system 200 may also be coupled to a calibration computing system 213 as discussed above in reference to the examples of FIGS. 1A, 1B, and 1C.

As illustrated in the example of FIG. 2B, the signal processing electronics 207 may include digital potentiometers (e.g., rheostats) (221, 223), instrumentation amplifier(s) 225, and one or more passive components (e.g., wires, resistors, and capacitors). Each sensor 227 in the sensor array 205 are configured to operate as “proximity sensors” and are arranged in a 2-dimensional array indexed one position at a time by the DEMUX 203. In some implementations of the multiplexer-based control system of FIG. 1A and the DEMUX-based control system of FIG. 2A, each “distance sensor” 227 in the respective array includes an infrared light emitting diode (IR LED) that projects infrared light. This projected light reflects off an object and is then sensed by a phototransistor in the sensor that allows a current to flow proportional in magnitude to the intensity of the reflected light received by the sensor. The intensity of the light received is proportional in magnitude to the distance and reflectivity of the object. The voltage output is then measured by an analog-to-digital converter (ADC) (e.g., a component of the signal processing electronics 207 or the prothesis controller 209) and the distance measured by the sensor can be calculated based on the measured voltage.

In some implementations, each sensor 227 of the array includes two resistors—one to set the voltage and wattage of the IR LED and the other to set the voltage and wattage of the phototransistor output. In the example of FIGS. 2A and 2B, only one sensor is powered at a time and, therefore, in some such implementations, the LED drains and transistor outputs of every sensor in the array 205 can be connected to the same two resistors. This decreases component and assembly costs. Additionally, in some implementations using a DEMUX 203 to selectively apply electrical power to individual sensors (or groups of sensors), only one instrumentation amplifier (INAMP) 225 is needed per DMUX sensor array as the voltages are large enough to preserve their integrity without a voltage follower circuit. The negative input of the INAMP is set to equal the minimum possible voltage of the sensor being measured. This makes the instrumentation amplifier output at the minimum sensor value close to 0v. This also removes much of the electrical noise from the output as this noise is on the sensor and the negative INAMP input. This negative INAMP input is set with a voltage divider that has a digital potentiometer (rheostat) 221 as one of its legs. The gain of the INAMP 225 is set so that the maximum INAMP output is close to a maximum ADC input (as determined, for example, during a calibration procedure). High-frequency noise is removed from the output of the potentiometers and the INAMP 225 with external resistors and/or capacitors.

Similarly, FIG. 1D illustrates an example of a filtering circuit for an implementation that uses a multiplexer 114 to selectively and successively couple the output signal from each individual sensor 227 in the sensor array to the output signal. Like the example of FIG. 2B, the filtering circuit in the example of FIG. 1D also includes an instrumentation amplifier (INAMP) 135 and a pair of digital potentiometers 131, 133.

The graph of FIG. 2C illustrates the operation of the signal filtering provided by the circuit in the example of FIG. 2B by graphing the unfiltered output signal of the two-dimensional array (signal 231), the noise component 233, and the filtered output signal with the noise component removed (signal 235). As shown in the graph of FIG. 2C, the operation of the filter circuitry removes the noise component and adjusts the amplitude of the output signal to fit a defined maximum amplitude and a define minimum amplitude (e.g., 5v and 0v, respectively, in the example of FIG. 2C).

In some implementations, control system, such as those illustrated in the examples of FIG. 1A, 1B, or 2A, may be adapted to operate selectively and interchangeably with one or more different commercially available prosthetic hands. FIG. 3 illustrates three examples of commercially available prosthetic hands that may be used with the control systems described in the examples above. The examples illustrated in FIG. 3 include Ottobock prosthetic hands and cosmetics, including from left to right, small system inner hand, small MyoHand VariPlus Speed, and medium Michelangelo hand. Although these specific examples are shown in FIG. 3, many other prosthetic devices may be used with and controlled by the control systems described herein. Furthermore, although examples presented in this disclosure may be described in reference to controlling a prosthetic hand, these examples can be adapted in other implementations to provide for muscle-movement-based control of other types of prosthetic devices (e.g., powered prosthetic arms or legs) and/or other non-prosthetic systems and actuators.

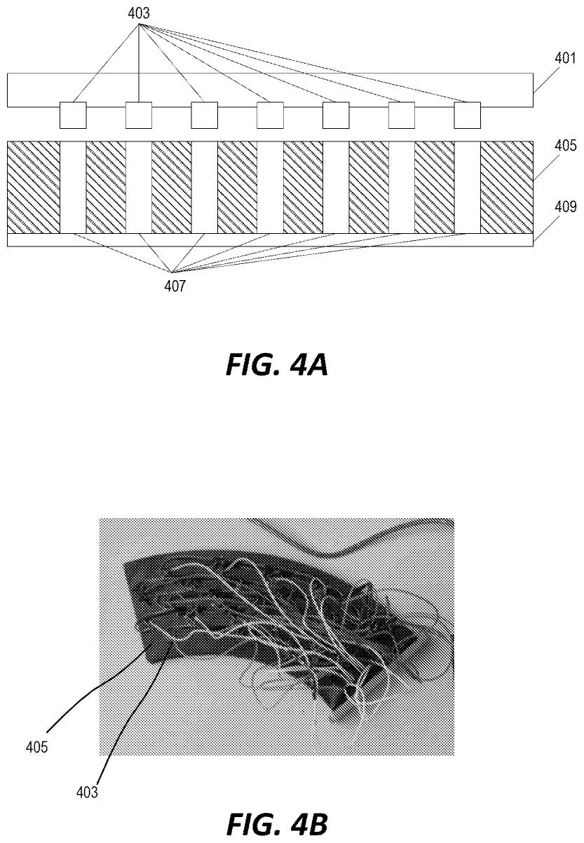

As described above, the control system operates by using a support structure to hold the two-dimensional sensors in place and positioning a compressible layer between the array and a tissue surface. FIGS. 4A and 4B illustrate two examples of such configurations. In the example of FIG. 4A, a plurality of distance sensors 403 are arranged in a two-dimensional array on a flexible, but incompressible, printed circuit board (PCB) layer 401. The array of sensors 403 is positioned adjacent to a compressible layer 405 formed of an opaque material (e.g., a compressible foam). A series of holes 407 are formed through the compressible layer 405 aligned with the position of each sensor 403 so that the light from each distance sensor 403 is able to pass through the compressible layer 405. A layer of reflective material 409 is positioned adjacent to the compressible layer 405 opposite the distance sensor 403. In this configuration, light is emitted from a sensor 403 into its respective hole 407 in the compressible layer 405, is reflected by the reflective layer 409, and the reflected light is detected by the sensor 403. In some implementations, the use of opaque material for the compressive layer ensures the distance measured by each individual sensor 403 is not affected by ambient light or light emitted by other sensors 403 in the array. However, in other implementations, partially or entirely translucent materials may be used for the compressible layer and the signal processing system may be adapted to detect muscle shape and/or movement based—not only on light reflected back to the sensor—but also based on how light from other sensors 403 in the array may be observed/sensed by a given sensor 403. In some implementations of the example of FIG. 4A, the sensors 403 may be affixed to the PCB layer 401. However, in other implementations, each individual sensor 403 may be embedded (or otherwise placed) in its respective hole 407 in the compressible layer 405 as shown in the example of FIG. 4B, which shows the sensors 403 and the compressive layer 405 without the support structure (e.g., PCB layer 401) in place.

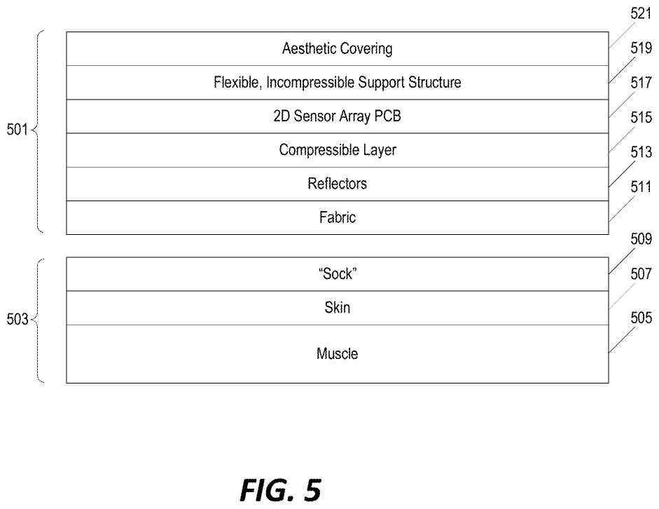

FIG. 5 illustrates one example of the two-dimensional array of distance sensors and the compressible layer integrated into a single wearable control device and also illustrates an example of how that wearable control device interfaces with the tissue surface to detect muscle movements. The example of FIG. 5 illustrates two separate layered structures—the layers of the wearable control device 501 and the layers corresponding to the user 503. In the example of FIG. 5, the wearable control device 501 is positioned on an external skin surface 507 of a user to detect movements (e.g., changes in shape) of the muscles 505 below the skin surface. In some implementations, a user may also wear a “sock” garment between the skin surface 507 and the wearable control device 501.

The wearable control device 501 includes a fabric layer 511 that is placed in contact with the skin surface 507 (or the sock 509) when the device 501 is worn and an aesthetic covering 521 enclosing the functional layers and components of the wearable control device 501. The wearable controller device 501 includes a two-dimensional array of distance sensors mounted to a flexible printed circuit board (i.e., PCB layer 517). The PCB layer 517 is positioned adjacent to a compressible layer 515 opposite a reflector layer 513. A flexible, incompressible support structure 519 is positioned between the PCB layer 519 and the aesthetic covering 521. In the configuration illustrated in FIG. 5, the layers of the wearable control device 501 are secured to the user 503 such that movements of the muscles 505 cause corresponding movements of the reflector layer 513 by compressing the compressible layer 513 while the distance sensors of the PCB layer 517 are held in place by the support structure 519. Accordingly, movements of the muscle 505 cause changes in the distance between the reflector layer 513 and each individual distance sensor.

It is noted that FIG. 5 presents just one example of a wearable control device 501 including a two-dimensional array of distance sensors and a compressible layer to monitor muscle movements/position. Other configurations are possible. For example, as discussed above, in some implementations, the wearable control device 501 does not include a reflector layer 513. Instead, the wearable control device 501 in other implementations may include an array of individual reflectors embedded into the compressible layer 513 or, alternatively, may expose the compressible layer 515 directly (or through one or more translucent materials) to the skin surface 507 so that light from each sensor is reflected directly by the skin surface 507.

Furthermore, although the example of FIG. 5 operates by moving the reflector layer 513 in response to muscle movements while the distance sensors remain stationary, in some other implementations, the position of the 2D sensor array and the reflector layer 513 may be reversed such that movement of the muscle 505 causes corresponding relative movement of each sensor in the 2D array by compressing the compressible layer 515 while the reflector layer 513 is held stationary by the support structure 519.

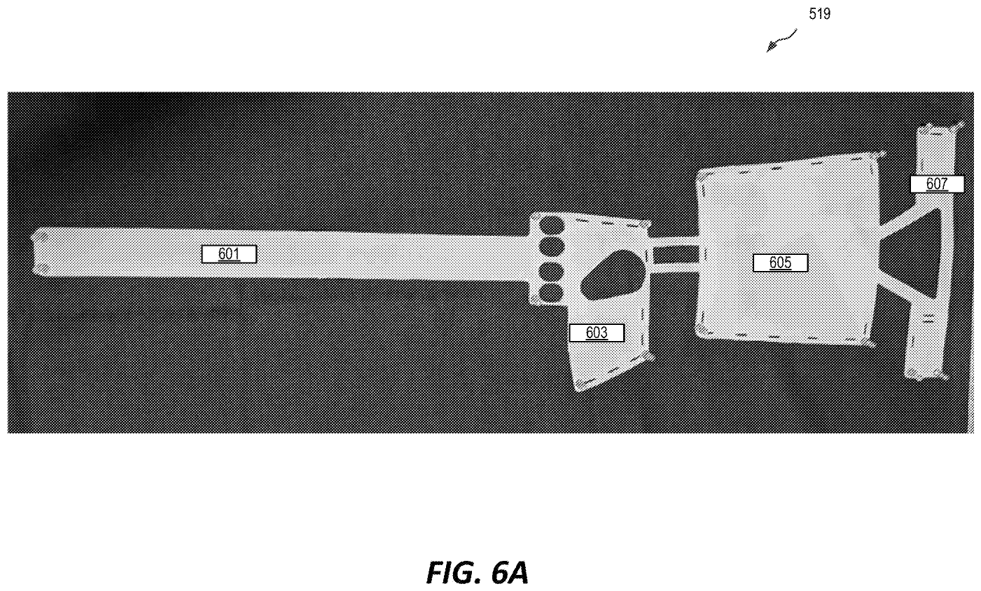

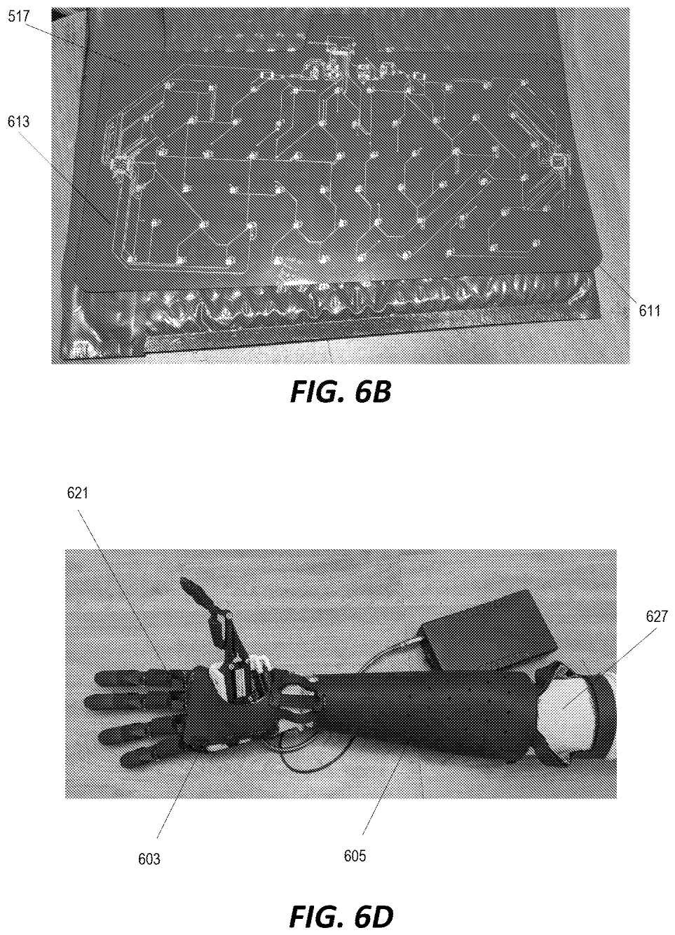

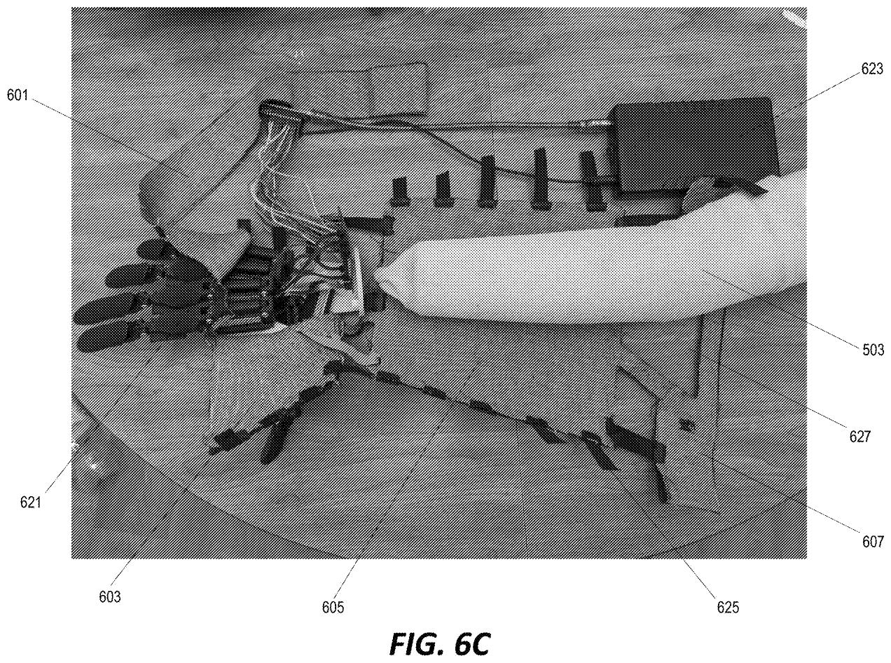

FIGS. 6A through 6D illustrate a specific example of a wearable control system including the layered arrangement illustrated in FIG. 5. In this example, the wearable control system is configured as a socket that selectively couples a prosthetic hand to an amputated arm. An example of the support structure 519 is shown in FIG. 6A. The support structure 519 is formed of a flexible material that is incompressible or significantly less compressible than the material of the compressible layer 515. In the example of FIG. 6A, the support structure 519 is laser cut as a two-dimensional form including a tongue 601 extending from a distal end of a fingerless glove section 603. The tongue 601 is configured to fold over the user's hand/arm to protect the skin surface from pinching or abrasion when the wearable control device is secured to the user's arm by straps (as described below). The fingerless glove section 603 includes holes for a thumb and each of four fingers and is sized to wrap around a hand (either a prosthetic hand or a user's actual hand) when the wearable control device is secured to the user's arm. A proximal end of the fingerless glove section 603 is coupled to a sensor array support section 605 that is configured to wrap around a forearm of the user and to hold the sensor array in place when the wearable control system is secured to the user's arm. The support structure 519 further extends to an upper arm support section 607 at its proximal end. As illustrated further below, the upper arm support section 607 in this example is used to secure the wearable control device to a user's upper arm for anchoring and includes a cut-away section that is to be positioned over the inner elbow of the user's arm to prevent pinching and irritation when the user's elbow joint bends. The support structure 519 includes additional holes cut into it to fix the distance sensors and the printed circuit board in place while allowing the distance sensors to see changes in muscle conformation. In some implementations, the grooves may be cut into the support structure 519 to facilitate bending.

FIG. 6B illustrates an example of the printed circuit board layer 517 of the wearable control device 503. In this example, 60 different distance sensors 611 are mounted to the surface of a flexible printed circuit board (PCB) 517 in a two-dimensional array pattern and are communicatively coupled by printed circuit traces 613. However, as noted above, the exact number of sensors and the arrangement of the sensors in the two-dimensional array may be adjusted/altered in other implementations. As also described in detail above, the sensors are either all turned on at the same time and read one-at-a-time using a multiplexer (as illustrated in FIG. 1A) or share a common output channel and are selectively powered on one-at-a-time using a demultiplexer (as illustrated in FIG. 2A).

The assembled wearable control system is illustrated in FIG. 6C. As discussed above in reference to FIG. 5, in the assembled wearable control system 503, the sensor array PCB layer 517 and the compressible layer 515 are housed within the aesthetic covering 521. In the example of FIG. 6C, both the aesthetic covering 521 and the fabric layer 511 on the underside are cut in the same shape as the support structure 519 such that the assembled device also resembles the shape of the support structure 519 include a tongue 601, a fingerless glove section 603, a sensor array support section 605, and an upper arm support section 607. In some implementations, the fabric layer 511 is affixed to the aesthetic covering 521 (e.g., by adhesive or by sewing) to encase the support structure 519, the compressible layer 515, the sensor array PCB layer 517, and the reflector layer 513. However, in other implementations, the aesthetic covering 521 and/or the fabric layer 511 are selectively removable for washing.

As shown in the example of FIG. 6C, the prosthetic hand device 621 is positionable in the fingerless glove section 603 of the wearable control system 503 by extending the fingers and thumb of the prosthetic hand device 621 through the applicable holes in the fingerless glove section 603. A prosthesis controller 623 is communicatively coupled to both the prosthetic hand 621 and the internal sensor array of the wearable control system 503.

In the example of FIG. 6C, the wearable control system also includes multiple straps 625 arranged along the outer edges of the fingerless glove section 603, the sensor array support section 605, and the upper arm support section 607. To secure the wearable control system to the arm 503 of a user, the tongue 601 is placed along the user's arm 503 (extending from the prosthetic hand 621 towards the user's elbow), the body of the wearable control system is wrapped around the arm (over the tongue 601), and the straps are used to secure the wearable control system in place around the user's arm 503. In the example of FIG. 6C, the straps 625 are provided as “hook-and-loop” straps. However, in other implementations, the wearable control system may be secured around the user's arm by other fasteners including, for example, lace, rivets, snaps, or toggles.

Furthermore, in some implementations, a series of holes are formed at the position of each strap 625 through the fabric layer 511, the support structure 519, and the aesthetic covering 521. Accordingly, in some such implementations, the layers of the wearable control system can be assembled by extending and securing the straps 625 through each of these holes in the different layers and can be disassembled (e.g., for washing of the fabric layer 511) by removing the straps 625.

FIG. 6D illustrates the wearable control system secured to the prosthetic hand 621 and the user's arm 503. As shown in FIG. 6D, when the wearable control system is secured to the user's arm 503, the sensor array section 605 is positioned along the palm-side of the user's forearm and extends by wrapping around the outer side of the forearm. FIG. 6D also shows the placement of the opening 627 adjacent to the inner elbow of the user's arm to allow for movement of the elbow joint without obstruction or causing irritation.

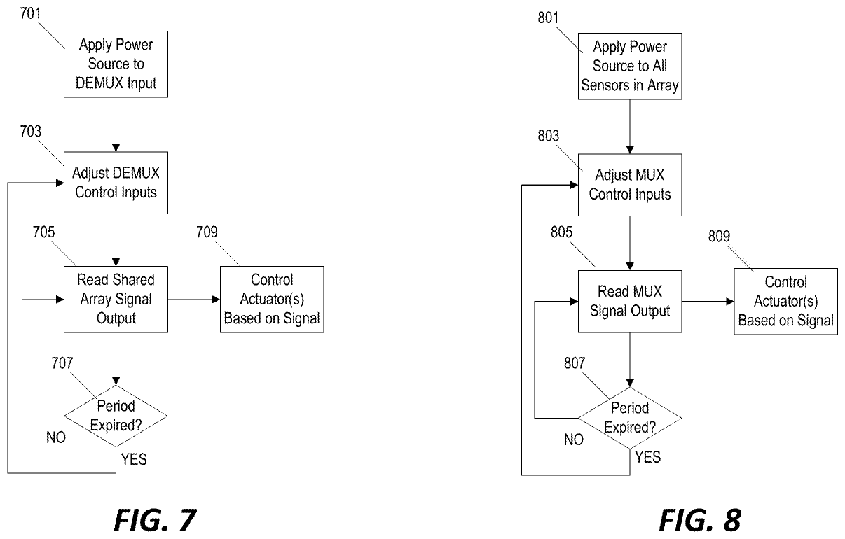

FIG. 7 illustrates an example of a method for controlling an actuator (such as, for example, the prosthetic hand 621 in the example of FIG. 6D) using a two-dimensional array of distance sensors with a shared output channel and a demultiplexer to selectively apply power to only one sensor in the array at a time (e.g., the control system of FIG. 2A). Electrical power from a power source is applied to the input line of the demultiplexer (step 701). The control input to the demultiplexer is adjusted by a controller (e.g., the prosthesis controller 209) (step 703) to cause the demultiplexer to selectively apply operating power from the power source to only one of the sensors in the array. The controller (e.g., the prosthesis controller 209) then reads the shared signal output channel of the array (step 705) and, after the expiration of a defined period of time (step 707), adjusts the control input provide to the demultiplexer (step 703) to cause the demultiplexer to apply operating power to a different sensor in the array. By controlling the demultiplexer in this way to sequentially applying operating power to each sensor in the array—one sensor at a time, a serialized signal is generated on the shared output channel of the array indicative of the muscle shape and movement. This serialized output signal is received by the controller (e.g., the prosthesis controller 209) and used to control the operation of an actuator (e.g., an actuator/motor of the prosthetic hand 211).

FIG. 8 illustrates an example of a method for controlling an actuator (such as, for example, the prosthetic hand 621 in the example of FIG. 6D) using a two-dimensional array of distance sensors with power continuously applied to all of the sensors and using a multiplexer to selectively adjust which sensor output is coupled to the output channel of the multiplexer. First, electrical power from a power source is applied to all of the sensors in the array (step 801). Because all of the sensors in the array are receiving operating power, every sensor in the array will also provide an output signal indicative of its measured distance to the multiplexer. A serialized output signal is then generated by periodically adjusting the control input to a multiplexer (steps 803 and 807). A controller (e.g., the prosthesis controller 118) (step 803) reads the serialized output signal at the output channel of the multiplexer (step 805) and controls the operation of an actuator (e.g., an actuator/motor of the prosthetic hand 120) based on the serialized output signal.

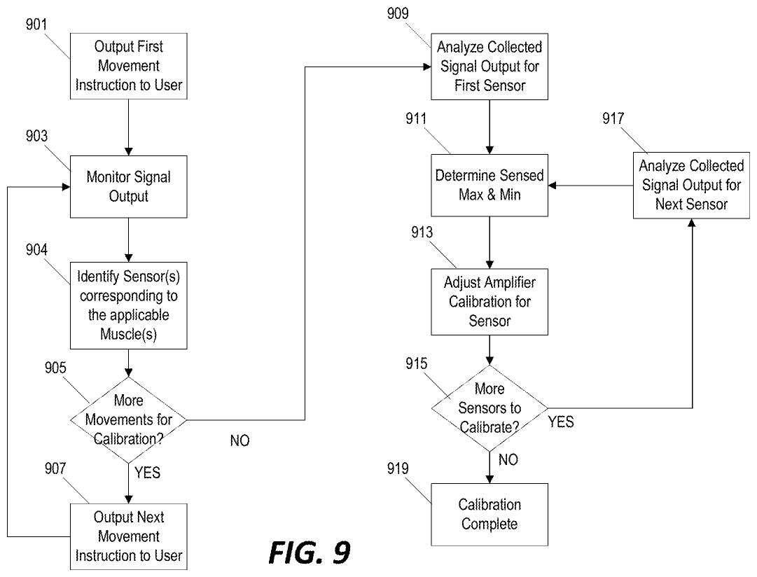

As discussed above in reference FIGS. 1A through 2B, a calibration computing system 122, 213 can be used to calibrate the control system for particular movements and to optimize the output of each sensor in the array based on observed signal outputs for each respective sensor. FIG. 9 illustrates an example of one such calibration process. First, the calibration computing system 122, 213 outputs a first movement instruction to a user (step 901). In some implementations, this instruction may be for the user to fully extend all fingers of a hand or to form a tight fist. The serialized signal output is monitored (step 903) and the controller analyzes the signal output to identify specific sensors that are positioned adjacent to the muscles (or muscle groups, areas, etc.) that change shape during the instructed “movement” (step 904). The specific sensor that is identified as corresponding to a muscle movement location for a particular gesture movement is referred to herein as the “indicative sensor” for that particular movement. This process for identifying the “indicative sensor(s)” is repeated for a series of additional movement instructions (step 907). When the signal output has been measured and the “indicative sensor(s)” have been identified for each prescribed movement in the calibration procedure (step 905), the collected signal corresponding to each individual sensor is analyzed (step 909) to determined a sensed maximum and minimum output value for the sensor (step 911). The calibration system then defines a gain and/or baseline voltage value for the sensor that causes the minimum voltage output value for the sensor to be close to the minimum analog input value for the analog-to-digital converter and to cause the maximum voltage output value for the sensor to be close to the maximum analog input value for the analog-to-digital converter (step 913). This is repeated for each sensor in the array (step 917). When all of the sensors in the array have been calibration (step 915), the calibration process is complete (step 919) and the control system is operated according to the gain settings and baseline voltage values defined for each sensor.

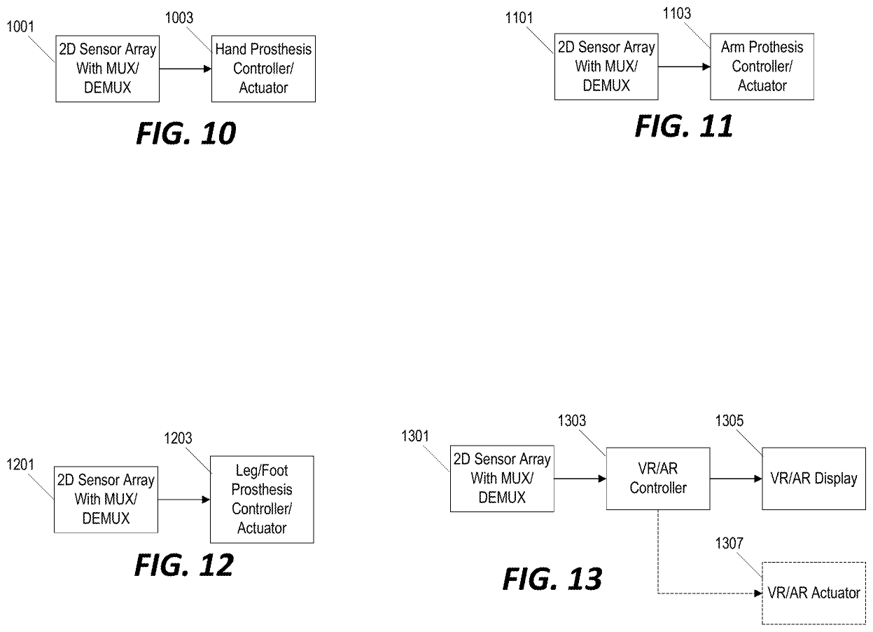

The methods and systems described in the examples above provide a control system positionable on a tissue surface of a user that uses a two-dimensional array of distance sensors and a compressible layer to monitor movements of muscles below the tissue surface. Systems and/or actuators are then controlled based on these sensed muscle movements. In several of the examples discussed above and as briefly illustrated again in FIG. 10, the system is adapted to position the sensor array 1001 to detect movements of muscles in a user's forearm corresponding to intended movements of fingers and the sensed output from the two-dimensional array of distance sensors is used to control the actuators of a powered hand prosthesis 1003. However, these systems can be adapted in other implementations to monitor different muscle groups and to control different systems.

For example, FIG. 11 illustrates an example in which the 2D sensor array (operated by a multiplexer or demultiplexer) 1101 is positioned on a chest of a user to monitor movements of pectoral muscles. The output of the sensor array system 1101 is then used to provide control signals for the operation of an arm prosthesis 1103. FIG. 12 presents another alternative example in which the 2D sensor array 1201 is positionable on a leg surface to monitor movements of leg muscles and the output of the sensor array system 1201 is then used to provide control signals for the operation of a leg or foot prosthesis 1203. For example, the sensor array might be positioned on a surface of the upper leg to monitor movements of the thigh muscle and to then control operation of a prosthetic knee based on the thigh muscle movements. Alternatively, the sensor array 1201 might be positionable on a lower leg surface to monitor movements of a calf muscle and to then control operation of a prosthetic ankle based on the calf muscle movements.

However, implementations of the 2D sensor array systems (such as those described above) are not necessarily limited to control of prosthetic devices in cases of amputation. Instead, the output signal of the 2D sensor array may be used to control other actuators or as a user interface input to other systems. For example, in a virtual reality system, a 2D sensor array 1301 may be positionable on a forearm of a user in order to track movement of a user's hand. This movement can then be used by the VR system controller 1303 as a user control input to the virtual reality system and images/interfaces displayed to the user on a VR display 1305 may be adjusted based on this user control input.

This movement tracking/detection functionality is also applicable to augmented reality (AR) systems in which movements of a user's hand, for example, are detected based on the output of the 2D sensor array 1301 and analyzed by an AR system controller 1303. In addition to using hand position as a user control input for adjusting the images and/or interfaces displayed to the user on an AR display 1305, the AR system controller 1303 may be further configured to control the operation of an actuator 1307 based on the determined hand position/movements.

Although the examples describe above focus specifically on measuring muscle movements (i.e., changes in muscle shape and thickness), in some implementations, additional functionality may be incorporated into the same system. For example, in some of the examples described above, the two-dimensional array of distance sensors includes a plurality of light-intensity distance sensors. In addition to measuring distances, some light-intensity distance sensors can also be operated to provide plethysmography and pulse oximetry functionality. Accordingly, in some implementations, the systems described herein can be further adapted to provide additional functions such as, for example, plethysmography and pulse oximetry with additional programming of the controller and, in some cases, without any additional components or circuitry. Similarly, in other implementations, the systems and structures described herein (including, for example, the layered configuration) can be configured to provide systems for plethysmography and/or pulse oximetry without including any muscle movement measurement of actuator/prosthesis control functionality.

Thus, the invention provides, among other things, systems and method for identifying bodily movements based on muscle movements measured at a tissue surface by a two-dimensional array of distance/proximity sensors configured to monitor the muscle movements based on compression of a compressible layer. Other features and advantages of this invention are set forth in the following claims.

What is claimed is:

1. A wearable sensor system comprising:
a compressible material positionable relative to a tissue surface;
a two-dimensional array of distance sensors, wherein the two-dimensional array of distance sensors is configured relative to the compressible material to detect compressive deformations of the compressible material; and
a support structure configured to hold the compressible material in place relative to the tissue surface such that muscle movements under the tissue surface cause the compressive deformations of the compressible material, and wherein the support structure is configured to restrict movement of the two-dimensional array during the muscle movements.

2. The wearable sensor system of claim 1 further comprising a controller configured to
receive one or more signals from the two-dimensional array of distance sensors indicative of the compressive deformation at a location of each distance sensor, and
determine a gesture operation based on the received one or more signals from the two-dimensional array of distance sensors.

3. The wearable sensor system of claim 2, wherein the wearable sensor system is configured to selectively couple to a forearm of a user such that muscle movements in the forearm cause the compressive deformations of the compressible material, and
wherein the controller is configured to determine a gesture operation based on the received one or more signals from the two-dimensional array by determining a hand movement corresponding to the muscle movements in the forearm.

4. The wearable sensor system of claim 3, wherein the controller is further configured to transmit a control signal to a powered hand prosthesis, wherein the transmitted control signal is configured to cause the powered hand prosthesis to move according to the determined hand movement.

5. The wearable sensor system of claim 4, further comprising a prosthesis socket configured to selectively couple the wearable sensor system to the forearm and to physically support the powered hand prothesis.

6. The wearable sensor system of claim 5, wherein the prosthesis socket includes a fingerless glove section sized to selectively couple the prosthesis socket to the powered hand prosthesis by receiving the fingers of the powered hand prosthesis through a series of spaced openings.

7. The wearable sensor system of claim 3, wherein the controller is further configured to transmit a control signal to an actuator, wherein the transmitted control signal is configured to cause the actuator to operate in a manner corresponding to the determined hand movement.

8. The wearable sensor system of claim 3, wherein the controller includes a virtual reality display controller, and wherein the virtual reality display controller is configured to adjust an interactive virtual reality environment in response to the determined hand movement as a user interface input.

9. The wearable sensor system of claim 2, wherein the wearable sensor system is configured to selectively couple to a leg of a user such that muscle movements in the leg cause the compressive deformations of the compressible material,
wherein the controller is further configured to transmit a control signal to a powered foot or leg prosthesis, wherein the transmitted control signal is configured to cause the powered foot or leg prosthesis to move according to the determined gesture operation.

10. The wearable sensor system of claim 2 further comprising a multiplexer,
wherein an output channel of each distance sensor is coupled to a different input channel of the multiplexer,
wherein the controller is configured to provide a control signal to the multiplexer causing the multiplexer to generate an output signal on the output channel of the multiplexer including serialized data indicative of distances sensed by each of a plurality of distance sensors in the two-dimensional array of distance sensors, and
wherein the controller is configured to receive the one or more signals from the two-dimensional array of distance sensors indicative of the compressive deformation at the location of each distance sensor by receiving the output signal of serialized data from the output channel of the multiplexer.

11. The wearable sensor system of claim 2 further comprising a demultiplexer,
wherein an input channel of the demultiplexer is culpable to a power source,
wherein each output channel of the demultiplexer is coupled to a power supply input of a different distance sensor in the two-dimensional array of distance sensors,
wherein the controller is configured to provide a control signal to the demultiplexer causing the demultiplexer to successively provide electrical power from the power source to each distance sensor in the two-dimensional array by providing the electrical power from the power source to only one distance sensor at a time,
wherein an output channel of each distance sensor in the two-dimensional array is coupled to a shared output channel of the two-dimensional array such that successively providing electrical power from the power source to each distance sensor generates an output signal on the shared output channel of the two-dimensional array including serialized data indicative of distances sensed by each of a plurality of distance sensors in the two-dimensional array of distance sensors, and
wherein the controller is configured to receive the one or more signals from the two-dimensional array of distance sensors indicative of the compressive deformation at the location of each distance sensor by receiving the output signal of serialized data from the shared output channel of the two-dimensional array.

12. The wearable sensor system of claim 11, further comprising a gain adjustment circuit configured to controllably adjust a gain of the output signal on the shared output channel of the two-dimensional array,
wherein the controller is further configured to
determine a maximum output signal value for each distance sensor in the two-dimensional array of distance sensors, and
operate the gain adjustment circuit to controllably adjust a gain of the output signal for each individual distance sensor when the electrical power is applied to the individual distance sensor by the demultiplexer,
wherein the controller adjusts a gain for the individual distance sensor by control the gain adjustment circuit to apply a gain setting to the output signal that would cause the determined maximum output signal value for the individual distance sensor to match a defined maximum signal value for the output signal.

13. The wearable sensor system of claim 2, wherein the gesture operation includes multiple degrees of freedom.

14. The wearable sensor system of claim 2, wherein the controller is configured to detect changed in muscle thickness and shape in response to the muscle movements.

15. The wearable sensor system of claim 2, wherein the controller is configured to
detect muscle force, muscle length, and muscle velocity based on the one or more signals from the two-dimensional array of distance sensors; and
operate a force and a velocity of at least one actuator based on the detected muscle force, the detected muscle length, and the detected muscle velocity.

16. The wearable sensor system of claim 1 further comprising a reflective layer positioned adjacent to the compressible material opposite the two-dimensional array of distance sensors,
wherein each distance sensor in the two-dimensional array of distance sensors is configured to emit light towards the compressible material and to detect an intensity of light reflected by the reflective layer.

17. The wearable sensor system of claim 1 further comprising a flexible printed circuit board layer, wherein the distance sensors of the two-dimensional array of distance sensors are mounted to the flexible printed circuit board layer, wherein the flexible printed circuit board layer, the compressible material, and the support structure are configured in a layered arrangement with the flexible printed circuit board layer positioned between the support structure and the compressible material.

18. The wearable sensor system of claim 1, wherein the two-dimensional array of distance sensors includes a plurality of light intensity distance sensors arranged in a two-dimensional array pattern.

19. The wearable sensor system of claim 18, wherein the two-dimensional array is curved to a contour of the tissue surface when the wearable sensor system is coupled to the tissue surface.
